SEQUENCE LISTING (1 sequence)

SEQ ID NO: 1 - protein, 39 aa, Artificial Sequence
  Sequence added to the FPS1 gene containing 3 HA epitopes
SGAIFYPYDVPDYAGYPYDVPDYAGYPYDVPDYAAQCGR

(19) United States
(12) Patent Application Publication
(10) Pub. No.: US 2012/0094294 A1
(43) Pub. Date: Apr. 19, 2012

(54) BMS1 PROTEIN EXPRESSION SYSTEM

(75) Inventors: Nicklas Bonander, Molndal (SE); Richard Darby, Hampshire (GB); Roslyn Bill, Solihull (GB); Ljuban Grgic, London (GB)

(73) Assignee: Aston University

(21) Appl. No.: 12/737,482

(22) PCT Filed: Jul. 17, 2009
(86) PCT No.: PCT/GB2009/001785

§ 371 Date: Jul. 11, 2011

(30) Foreign Application Priority Data
Jul. 18, 2008 (GB) 0813253.2

(51) Int. Cl. C12Q 1/68; C12P 21/00; C12N 5/10; C12N 15/81; C12N 15/85; C12N 15/82; C12N 1/19; C12N 15/79

(52) U.S. Cl. 435/6.12; 435/254.2; 435/348; 435/349; 435/325; 435/254.21; 435/254.23; 435/69.1; 435/320.1; 435/419

(57) ABSTRACT

Eukaryotic cells having upregulated BMS1 genes and a nucleotide sequence encoding a recombinant protein or fragment for the production of such proteins or fragments are provided.

This is based on the finding that elevated BMS1 levels, improve yields of recombinant proteins.

Methods of producing recombinant proteins using such cells are also provided.

The invention relates to methods for improving production of recombinant proteins in eukaryotic cells and to cells for use in such methods. In particular, the cells over express BMS1 and are preferably yeast cells.

The overproduction of proteins such as eukaryotic membrane proteins continues to be a contemporary challenge in structural biology as it is plagued by low yields. Since the vast majority of membrane proteins are not abundant enough from natural sources, recombinant overproduction is a requirement to move the field forward. Although more structures of bacterial membrane proteins are emerging, only five structures have been obtained for recombinant eukaryotic membrane proteins. Of these five, three were produced in the yeast Pichia pastoris¹⁻³. To date, this has been the yeast species of choice, but recently attention has turned to Saccharomyces cerevisiae as it offers several advantages over P. pastoris not least a raft of verified molecular- and micro-biological tools including a freely-available genome sequence and several deletion strain collections. Importantly, this yeast has been used to produce several membrane proteins in high yields⁴ including the rabbit Ca2+ ATPase⁵, which was subsequently crystallised and yielded data that was consistent with the structure that had been derived from naturally-abundant material⁶.

Despite the fact that focusing on promoters and expression tags is not sufficient to overcome the complex problem of inserting a recombinant membrane protein into the membrane of any heterologous host, this is still the most widely used strategy in the vast majority of protein production experiments. Two systematic possibilities exist for approaching the problem in a rational manner: engineering the protein to make it more amenable to production, or understanding the host response in order to engineer improved strains. Recently, Martinez Molina and co-workers³ used in vitro evolution to optimise membrane protein yields in E. coli. The Inventors and others⁸ have taken a more systematic approach to optimising the host response in and S. cerevisiae, and recently similar studies have been done in E. coli, but this knowledge has not been exploited to gain an understanding of the critical parameters in successful membrane protein production experiments for the generation of new strains for structural genomics projects.

The strains were initially identified using the well-characterized⁹⁻¹¹, eukaryotic glycerol facilitator, Fps1, which based on previous experience, is a non-trivial production target for further structural study⁷. Bonander et al⁷ discloses a study to try and identify proteins associated with membrane protein production. 24 genes of known function and 15 genes of unknown function were identified as having changes in expression in yeast under different growth conditions. Rapid growth conditions were not suitable for the production of membrane proteins, but rather tightly controlled conditions were required for optimised protein production. On generating deletion or overexpression strains guided by the transcriptome analysis of high yielding conditions, many were found not to improve recombinant protein yields. Examples include a SEC62 overexpression strain and an erg 9 deletion strain. SRP102 was found to be down-regulated in high yielding experiments but srp102 deletion strains gave only wild type yields of Fps1.

Developing from this, a screen identified spt3Δ, srb5Δ and gcn5Δ as effective production hosts not only for Fps1, where the yield improvement was up to 69-fold over the corresponding wild-type control, but also the human G-protein coupled receptor, adenosine 2A Receptor (hA2aR), for which the Inventors improved the functional yield 4-fold compared to wild-type. Improved yields of Fps1 were not strongly correlated with promoter- or FPS1 transcript number, but a translational mechanism was confirmed by the observation that each strain had elevated levels of BMS1 transcript compared to wild-type. Subsequent overexpression of BMS1 in a doxycycline-dependent manner revealed that maximal membrane protein yield is correlated with an optimum level of BMS1 transcript for Fps1 and hA2aR. This is related to the efficiency of translation as measured in polysome profiling experiments and quantitated by flow microcalorimetry.

The Inventors have unexpectedly found that BMS1 upregulation can improve the production of recombinant proteins in eukaryotic cells such as yeast. The Inventors investigated the production in selected strains of yeast and identified and characterised the common factor, BMS1 upregulation, resulting in improved recombinant protein production.

The invention provides a eukaryotic cell comprising an upregulated BMS1 gene or a functional fragment thereof and a nucleotide sequence encoding a recombinant protein or a fragment thereof.

The Inventors have found that upregulating the BMS1 gene improves the production of the recombinant protein.

The term “upregulated” means that the BMS1 gene product is provided in higher concentrations than in a wild-type eukaryotic cell.

The term “functional fragment” of the BMS1 means that the gene may comprise one or more deletions, insertions or other modifications whilst still retaining BMS1 activity to aid improved production of the recombinant protein or a fragment thereof.

A fragment of the recombinant protein may be encoded by the nucleotide sequence. For example, the recombinant protein may still retain a particular activity, such as enzymatic activity, or receptor or ligand binding, that is found in the native protein. It may also be, for example, an antigenic fragment of a protein.

The recombinant protein may be a fusion protein of two or more different proteins or fragments.

The recombinant protein may be a protein naturally found in the cell, operatively attached to, for example, a different regulatory sequence, such as a promoter, to that normally found with, or operatively linked to, that protein. Alternatively, the recombinant protein may be a protein not naturally found in the cell.

The term “operatively linked” means that, for example, a nucleotide sequence encoding a promoter is linked to a nucleotide sequence encoding a protein in such a way that the promoter controls expression of the nucleotide sequence encoding the protein.

BMS1 is an essential nucleolar protein conserved throughout the eukaryotic kingdom. It has been suggested as having a regulatory role in the biogenesis of the 40S ribosome subunit as well as being a GTP-binding protein^{12, 13} BMS1 is proposed to act in a similar manner to other GTPases acting as a “molecular switch” to regulate the biological pathway.

Strains of yeast lacking BMS1 are not viable. In Saccharomyces cerevisiae it has been identified to be present on chromosome XVI, 143170 to 139619. Similar genes have been identified in a broad range of organisms, including mouse (Uniprot acc. number Q6ZQHO), Schizosaccharomyces pombe (S. pombe—Q 94653), Yarrowia lipolytica (Q6CB53). Kluyveromyces lactis (Q6CWR6), Eremothecium gossypii (Q755D6), Candida glabrata (Q6FV00), Drosophila melanogasta (Q9VVC9), Anopheles gambiae (Q7Q6F7), Arabidopsis thaliana (Q9M9Y0) and human (Q14692).

The upregulated BMS1 gene may be upregulated by one or more of
(i) A mutation in a regulatory sequence of the BMS1 gene. For example, this may be in the promoter linked to the BMS1 gene or a nucleotide sequence associated with regulation of the promoter;
(ii) A mutation in the BMS1 gene resulting in increased activity of the BMS1 product;
(iii) By having increased numbers of the BMS1 gene in a cell. This can be achieved, for example, by increasing the copy number of the BMS1 gene by providing additional copies on, for example, plasmids or other vectors;
(iv) Providing in the cell one or more BMS1 genes and/or actively linked to a constitutive promoter;
(v) Providing in the cell one or more BMS1 genes operatively linked to an inducible promoter.

Constitutive and inducible promoters which may be attached to the BMS1 gene are generally known in the art for different eukaryotic cells. They are preferably different to the naturally occurring BMS1 promoter. Suitable promoters are available in a wide variety of suitable cloning vectors which may be selected according to the eukaryotic cell. Plants may use, for example, Agrobacterium Ti plasmids. Insect cells may utilise baculovirus based vectors. Avian and mammalian cells may use viral vectors, such as SV40, alternatively, the nucleotide can be integrated in the genome of the cell.

For example, the constitutive promoter, CaMV35S promoter has been used in a wide variety of cells. Rice actin promoters have been used in plant cells. Yeast promoters include PGK1 and PYK1 which may be included by glucose. Copper inducible promoters, such as CUP1, are also known in yeast. Steroid inducible promoters for use in the over expression of genes in eukaryotes have been also described. A tet0 promoter may be used for yeast cells, or, for example AOX1, GAL1 or the TPI promoter.

The Inventors have found that increased yields of recombinant proteins can be produced by an optimum level of the BMS1 transcript. Hence, the cell is preferably selected to have an optimised level of BMS1. Typically, more than a four fold increase of BMS1 levels does not lead to much more improvement in expression in the constructs so far tested.

This may be achieved, for example, by selection of a particular mutant of the eukaryotic cell, by changing BMS1 copy numbers, or by regulating the expression of the BMS1 gene. This latter regulation may be achieved by optimising growth conditions or by using an inducible promoter. The selection of an inducible promoter means that levels of BMS1 can be varied by using different amounts of an inducing compound, for example, in the growth media of the cell.

The Inventors have found that optimum BMS1 concentrations vary from protein to protein. For example, for S. cerevisiae, preferred levels of BMS1 are up to four fold over wild type for those strains currently tested. Hence BMS1 levels may be 1.5, 2.0, 2.5, 3, 3.5, 4, 4.5, 5 fold increased over wild type levels.

The eukaryotic cell is preferably a fungus, such as a yeast. However, because of the ubiquitous nature of BMS1 in other eukaryotes, it is expected that BMS1 will also be important in other cells. These include plants, including algae, lower order plants, monocotyledous and dicotyledous plants, including oats, wheat, barley, maize, rice, Nicotiana, Arabidopsis, Solanum and Brassica. The cell may be from a crustacean, an insect, a reptile, an amphibian, a bird or a mammal, including mice, rats and humans.

The cell may be a single type of cell or be part of a tissue, seed or whole organism.

Preferably the eukaryotic cell is from a yeast such as Saccharomyces, Schizosaccharomyces or Pichia.

Preferred yeasts are Saccharomyces cerevisiae, Schizosaccharomyces pombi and Pichia pastoris, most preferably S. cerevisiae, though other yeasts are expected to behave the same.

The strains may be spt3Δ, srb5Δ and gen5Δ or for example a BMS1 overexpression strain.

Preferably the promoter operatively linked to BMS1 is the tetO promoter. It is preferably repressed with doxycycline.

The recombinant protein may be any recombinant protein that occurs naturally in the cell or non-naturally in the cell. It may be soluble or a membrane protein. It may be produced from a suitable vector encoding the nucleotide sequence or from a nucleotide sequence integrated into the genome of the cell.

Preferably the protein is a GPCR. Other proteins, such as luciferase or GFP (green fluorescent protein) may also be used, to study the effect of BMS1 on expression.

Other proteins include antibodies, trypsinogen, serum albumin, lipoprotein lipase and erythropoietin, or fragments thereof.

G protein coupled receptors (GPCRs) are also known as seven transmembrane receptors (7TM's). This is a large protein family of transmembrane receptors that detect molecules outside the cell and activate signal transduction pathways with the cell. GPCRs are found throughout the eukaryote kingdom. They are implicated in many diseases and are the target of around half of all modern medicinal drugs. Hence there is considerable interest in being able to express them to study them.

Binding of an external signal to a GPCR usually creates a conformational change in the receptor, causing activation of a G protein. G proteins act as molecular switches, alternating between an inactive guanosine diphosphate (GDP) and active guanosine triphosphate (GTP) boundstate, ultimately going on to regulate downstream cell processes.

GPCRs include receptors for sensory signal mediators (e.g. light and olfactory stimulatory molecules); adenosine, bombesin, bradykinin, endothelin, γ-aminobutyric acid (GABA), hepatocyte growth factor, melanocortins, neuropeptide Y, opioid peptides, opsins, somatostatin, tachykinins, vasoactive intestinal polypeptide family, and vasopressin; biogenic amines (e.g., dopamine, epinephrine, norepinephrine, histamine, glutamate (metabotropic effect), glucagon, acetylcholine (muscarinic effect), and serotonin); chemokines; lipid mediators of inflammation (e.g., prostaglandins, prostanoids, platelet-activating factor, and leukotrienes); and peptide hormones (e.g. calcitonin, C5a anaphylatoxin, follicle-stimulating hormone (FSH), gonadotropic-releasing hormone (GnRH), neurokinin, thyrotropin-releasing hormone (TRH), and oxytocin. GPCRs that act as receptors for stimuli that have yet to be identified are known as orphan receptors.

Preferred GPCRs include adenosine 2a receptor, kappa-type opioid receptor, neuromedin K receptor muscarinic (m4) receptor, vasopressin receptor, an aquorin receptor (such as AQP1 or AQP4) and dopamine D2 receptor.

Another protein which may be used is human aquaporin, AQP6.

The invention also provides a method of producing a recombinant protein or a fragment thereof from a nucleic acid sequence encoding the recombinant protein or fragment, comprising providing a eukaryotic cell having upregulated BMS1 and expressing the nucleic acid sequence encoding the recombinant protein, or a fragment thereof, in the cell.

Preferably, the levels of the BMS1 transcript are optimised for production of the recombinant protein.

The methods of the invention preferably use a cell as previously described above.

The ability to identify suitable candidates of cells for expressing recombinant proteins is important. Accordingly, the invention also provides methods for testing for eukaryotic cells suitable for being used to produce recombinant proteins by testing for BMS1 expression. This may be an antibody based test, to test for levels of the BMS1 product or alternatively using, for example, real time PCR to test for the levels of BMS1 expression.

Cells for use in the methods of the invention comprising a BMS1, or a functional fragment, operatively linked to a heterologous constitutive or heterologous inducible promoter are also provided. That is, the gene may be attached to a non-native promoter not normally attached to the BMS1 gene. Cells and promoters may be as defined above.

Kits for the production of recombinant proteins comprising (i) a vector encoding BMS1, or a functional fragment thereof, preferably attached to a heterologous constitutive or heterologous inducible promoter, and (ii) a nucleotide sequence encoding a recombinant protein or a fragment thereof, are also provided. The nucleotide sequence may be on the same or a different vector.

Kits may also be provided comprising a modified cell, such as a yeast, having elevated levels of BMS1 expression. Such cells may be as defined above.

Vectors, such as plasmids are generally well known in the art and will depend on the cell type used. This is readily assessed by the skilled person. Suitable promoters and vectors for the BMS1 protein and recombinant protein may be as defined above.

The Inventors have also identified that higher levels of expression of SRB6 can also improve levels of expression of recombinant proteins.

Accordingly, the eukaryotic cells of the invention may additionally comprise upregulated SRB6 genes or functional fragments thereof. Such upregulated levels may be 1.5, 2.0 or more fold increased over wild type. Cells may be selected for increased SRB6 expression. Alternatively, an SRB6 gene or functional fragment thereof may be operatively linked to a heterologous constituitive or heterologous inducible promoter, in a similar manner to that described above for BMS1. Kits comprising SRB6 genes or functional fragments are also provided.

Expression of SRB6 may be optimised for production of recombinant proteins.

The invention will now be described by way of example only with reference to the following figures

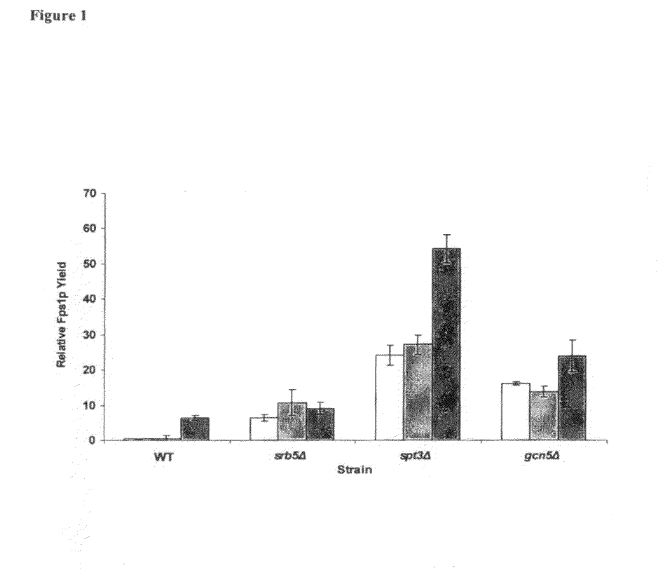

FIG. 1. Analysis of yields of Fps1 from the three selected deletion strains cultured in 2 L bioreactors. Fps1 yields are reported relative to a control sample, which previously represented our highest Fps1 yield obtained, as described in the Methods section. Maximum yields are indicated from four immunoblots from duplicate fermentations. Error bars represent the standard deviation (n=4). White bars are the values for cells grown in CSM medium, pale grey for CSM medium supplemented with 10 μg/ml myo-inositol and dark grey bars for 2×CBS medium.

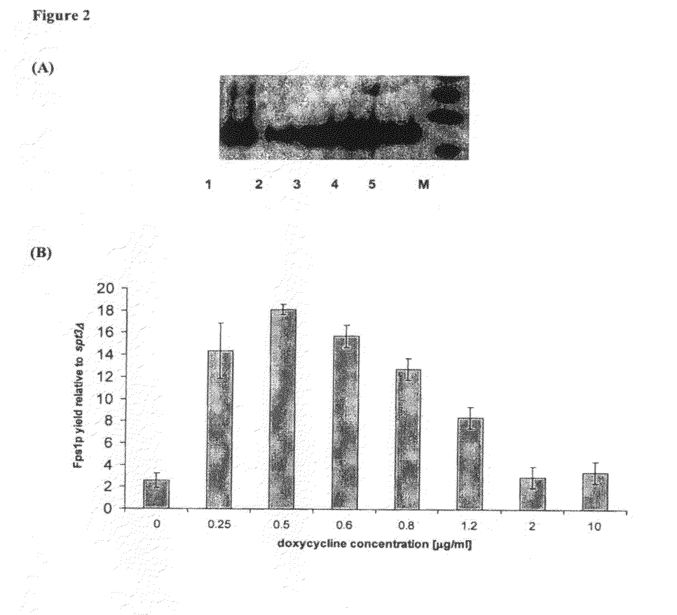

FIG. 2. Maximum yields of Fps1 are achieved by tuning BMS1 transcript number in a doxycycline-repressible system (yTHC). (A) Gel showing Fps1 yields for 75 μg total wild-type membrane (lane 1), and 15 μg total yTHCBMS1 membranes extracted from cultures grown in the presence of 0 (lane 2), 0.25 (lane 3), 0.5 (lane 4) and 10 μg/mL (lane 5) doxycycline. The markers (M) are 62, 98, 188 kDa. (B) Fps1 yield in shake flasks is presented for the yTHCBMS1 overexpression strain relative to that in the spt3Δ strain. Error bars represent the standard deviation (n=2).

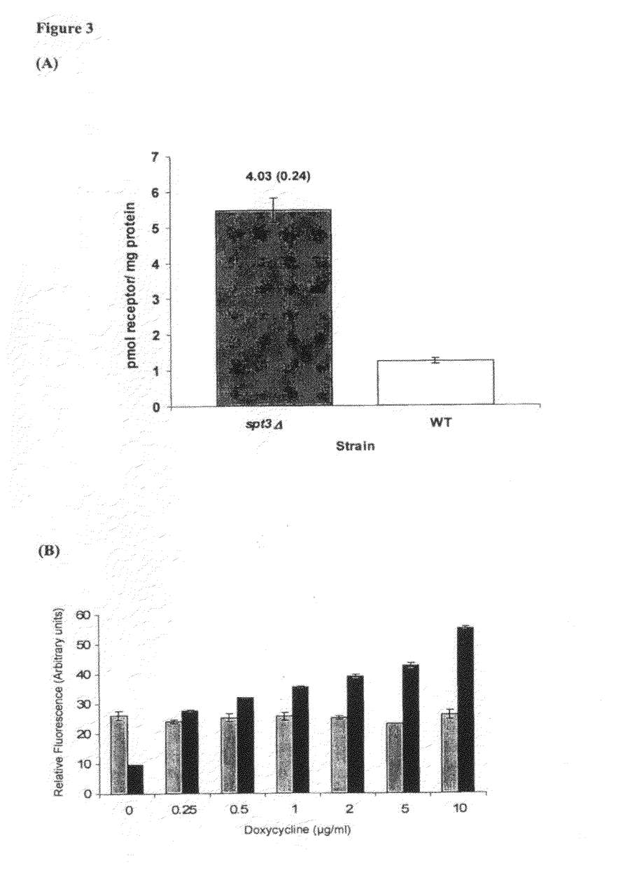

FIG. 3. The spt3Δ and the yTHCBMS1 strains, which give the highest yields for Fps1, can also be used to improve the functional yield of other proteins. (A) Binding of theophylline to yeast membranes producing the G-protein coupled receptor hA2aR is shown for duplicate determinations, error bars indicate the standard error of the mean (n=2). The value above the bar is the fold increase in yield over wild-type as determined by immunoblots. The standard deviation is given in parenthesis, where n=4. (B) The relative fluorescence of GFP per OD₆₀₀ unit of yeast is shown for triplicate determinations with varying concentrations of doxycycline. The grey bars represent wild-type data and the black bars represent yTHCBMS1 data. The error bars indicate the standard error of the mean (n=3).

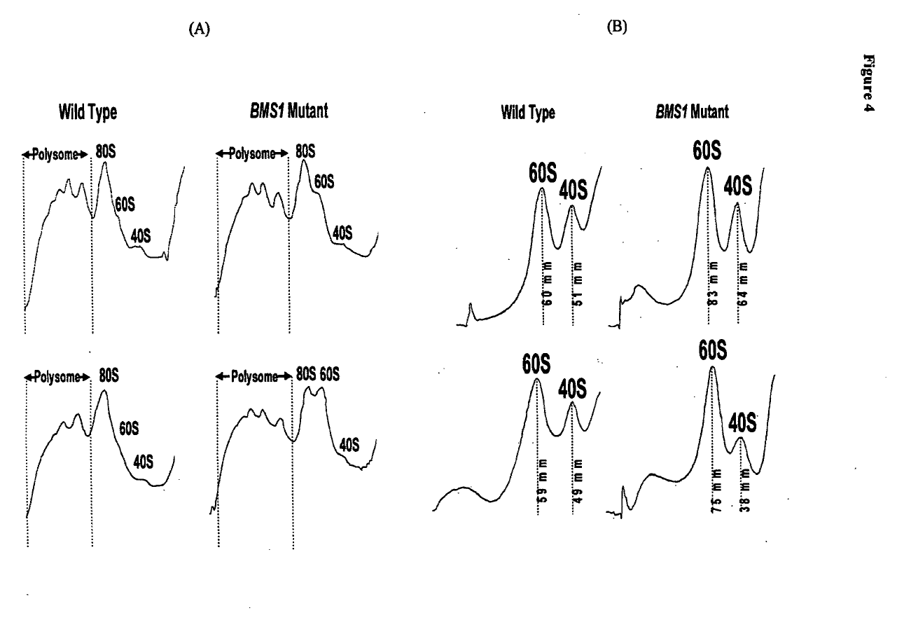

FIG. 4. Analysis of the BMS1 strain. (A) Polysome OD₂₅₄ profiles for yeast strains expressing Fps1 in the absence (upper panels) or presence (lower panels) of 0.5 μg/mL doxycycline. (B) Ribosome dissociation profiles at OD₂₅₄ for 100 mM EDTA treated samples of wild-type and yTHCBMS1 strains expressing Fps1 in the absence and presence of 0.5 μg/ml doxycycline (upper and lower panels respectively).

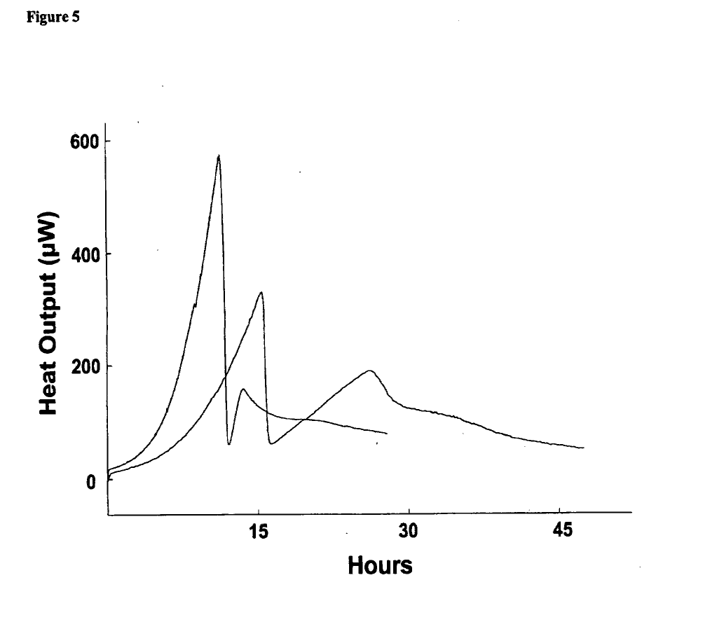

FIG. 5. Thermodynamic profile of independent bioreactor cultures of wild-type in the presence of 0.5 μg/ml doxycycline expressing Fps1 (low yielding condition; heavy black trace) and yTHCBMS1 in the presence of 0.5 μg/ml doxycycline expressing Fps1 (high-yielding condition; grey trace). Both strains in the absence of 0.5 μg/ml doxycycline expressing Fps1 overlay the black trace exactly (not shown for clarity).

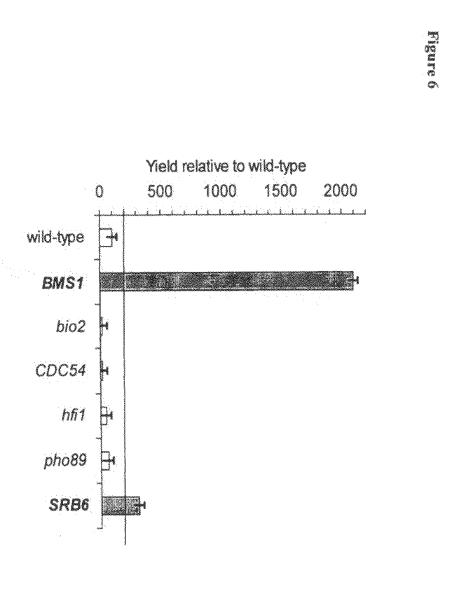

FIG. 6 shows analysis of Fps1 yields showing SRB6 and BMS1 improve Fps1 expression.

### METHODS

Vectors. The FPS1 gene was tagged at its 3′ end replacing the carboxy-terminal threonine residue with a sequence encoding three HA epitopes to permit immunodetection: SGRIFYPYDVPDYAGYPYDVPDYAGYPYDVPDYAAQCGR. The HA sequences are underlined. The construct was expressed from the TPI promoter in the 2μ pYX212 or pYX222 yeast expression vector (Novagen) which contain the URA3 or HIS3 selection marker respectively other yeast expression vectors may also have been used. The gene was cloned into the Bam H1 and Hind III sites and the vector transformed into S. cerevisiae using the lithium acetate method.

The hA2aR construct was PCR amplified introducing Hind III and Sal I sites from the pPICZA-FH-dG hA2aR vector kindly provided by Dr Niall Fraser, the construction of which is detailed in Protein Expr Purif 49 129-137 (2006). The gene was cloned into the Hind III Sal I sites of pYX212 and pYX222 and the vectors transformed into S. cerevisiae using the lithium acetate method.

The GFP construct was PCR amplified from pGFPuv (CloneTech) introducing a Hind III site and the yeast alpha-secretion factor at the 5′ end and a Xba I site at the 3′ end. The gene was cloned into the Hind III Xba I sites of pYX212 and pYX222 and the vectors transformed into S. cerevisiae using the lithium acetate method.

The yTHCBMS1 strain is available from Open Biosystems and is part of the collection which contains 800 essential yeast genes for which expression is regulated by doxycycline. The endogenous promoter has been replaced with a Tet-titratable promoter in the genome. Thus, the expression of the gene can be switched off by the addition of doxycycline to the yeast's growth medium. The yTHC mutant strains are haploid MATa strain R1158 created from the background strain BY4741 by a one-step integration of the tTA transactivator, under the control of the CMV promoter, at the URA3 locus. A plasmid carrying a kanR-tetO7-TATA cassette was then integrated into the genome replacing the endogenous promoter. Without doxycycline in the media the Tet-promoter is fully activated. Addition of doxycycline in a titratable manner allows for down regulation of the promoter until the gene of interest is no longer expressed at detectable levels. The optimal condition for each strain needs to be determined empirically. The genotype of wildtype R1158 is—URA3::CMV-tTA MATa his3-1 leu2-0 met15-0

Yeast strains and culturing conditions. S. cerevisiae strain BY4741 was the parental strain for the deletion mutants spt3Δ, srb5Δ and gcn5Δ publicly available from the EUROSCARF collection (web.uni-frankfurt.de/fb15/mikro/euroscarf). Yeast cells were cultured in 2.5 L bioreactors containing 2 L of either CSM f myo-inositol or 2×CBS. CSM was composed of 1.7 g/L yeast nitrogen base (YNB) without amino acids, 5 g/L ammonium sulphate supplemented with 2% glucose±10 μg/ml myo-inositol, 2×DO solution minus histidine or uracil (www.clontech.com/images/pt/PT3024-1.pdf) and 10 mM MES pH 6. 2×CBS was composed of 10 g/L ammonium sulphate, 6 g/L potassium dihydrogenphosphate, 1 g/L magnesium sulphate supplemented with 2% glucose, 2 ml/L of trace element solution and vitamin stock (recipes shown below), 2×DO solution minus histidine or uracil. The pH was adjusted to, and maintained at, 6 via the online addition of 0.5 M NaOH. The agitation, aeration and temperature of the cultures were maintained at 700 rpm, 1 L per minute and 30° C. respectively. 1 L trace element solution was composed of the following: 15 g EDTA, 4.5 g ZnSO₄.7H₂O, 1 g MnCl₂.4H₂O, 0.3 g CoCl₂.6H₂O, 0.3 g CuSO₄.5H₂O, 0.4 g Na₂MoO₄.2H₂O, 4.5 g CaCl₂.2H₂O, 3 g FeSO₄.7H₂O, 1 g H₃BO₃ and 0.1 g KI. The pH was maintained at 6.0 with 1 M NaOH throughout the addition and finally adjusted to pH 4 with 1 M HCl prior to autoclave sterilisation and storage at 4° C. 1 L vitamin solution was composed of the following: 0.05 g D-biotin, 1 g Ca D(+) panthothenate, 1 g nicotinic acid, 25 g myo-inositol, 1 g thiamine hydrochloride, 1 g pyridoxol hydrochloride and 0.2 g D-amino benzoic acid. pH maintained at 6.5 with 1 M HCl. The vitamin solution was filter sterilised and stored as 20 ml aliquots at 4° C. Plasmid retention was verified by the plating of the cells onto CSM+inositol agar in the absence of histidine and incubation at 30° C. for 4 days. To initiate each experiment, 200 mL of a given medium were inoculated with fresh yeast cells in a baffled shake flask and cultured over the weekend in a shaking incubator at 30° C., 220 rpm. This pre-culture was subsequently used to inoculate the bioreactors to initial OD₆₀₀=0.05.

Sampling, extracellular substrate determination and membrane preparation. Samples were withdrawn at various points in both the glucose and ethanol phases. 15-100 mL culture were centrifuged at 5000×g, 4° C. for 5 min. 0.5 mL of the supernatant was stored at −20° C. for glucose and ethanol analyses. The cell pellets were frozen in liquid nitrogen and stored at −80° C. for subsequent membrane preparation. Ethanol analysis (10176290035, R-Biopharm, Darmstadt, Germany) was performed according to the manufacturer's instructions. Glucose concentrations were calculated with an Accu-Chek Active glucose analyzer (Roche Diagnostics, Burgess Hill, UK). Cell pellets were fractionated with glass beads (1:1 ratio) in 2 mL cell breaking buffer (50 mM sodium phosphate pH 7.0, 2 mM EDTA, 5% glycerol w/v 2 mM PMSF). The cells were agitated in a Fast Prep (Thermo Scientific) at speed 6.5, employing 6×45 sec pulses with 2 min incubations on ice between pulses. The samples were clarified at 10,000×g, 4° C. for 30 min and the total membrane pellet recovered from the supernatant at 100,000×g, 4° C. for 60 min. Total membranes were re-suspended in 50 μL of Buffer A (20 mM Hepes pH 8, 50 mM NaCl, 10% glycerol w/v) and the protein concentration determined using a Bio-Rad (Hemel Hempstead, UK) Bradford-based assay with bovine serum albumin as standard.

Immunoblotting and yield analysis. 30-100 μg of total membranes were loaded per lane on an 8% polyacrylamide gel and separated by SDS-PAGE at 150 V for 1.25 h. Proteins were subsequently transferred to a nitrocellulose membrane (ProTran, Geneflow, Fradley, UK) at 100 V for 1 h. The membrane was blocked with phosphate-buffered saline (PBS) containing 5% milk overnight at 4° C. before incubating with mouse monoclonal anti-HA (clone 12CA5; Roche) at a 1:5,000 dilution in PBS/5% milk for 1 h at room temperature with gentle agitation. The membrane was subsequently washed twice with PBS/0.2% Tween 20 for 5 min before incubating with goat anti-mouse horseradish peroxidise-conjugated secondary monoclonal antibody (Sigma-Aldrich, Gillingham, UK) at a 1:5,000 dilution in PBS/5% milk for 60 min at room temperature with gentle agitation. The membrane was washed as above and developed using an enhanced chemiluminescence detection kit (Geneflow) following the manufacturer's instructions and visualized with a Chemidoc (UVltech, Cambridge, UK). The signal from each lane was quantified using either UVIband or the ImageGauge programme and was expressed relative to our internal control (which gave the same yield of Fps1 per mg of total membrane as our previously-published control⁷) and was corrected for the amount of total membranes loaded per lane.

RNA preparation and real time quantitative PCR. Yeast cells (60 mL) from two biological replicates and two technical replicates were harvested and frozen in liquid nitrogen. Total RNA was then prepared using the RNeasy kit from Qiagen with on-column DNAse treatment, following the manufacturer's instructions. Analysis of mRNA was performed using real time quantitative PCR (Q-PCR). 1.1 μg RNA was used in the cDNA reaction using the iScript cDNA Synthesis Kit (Bio-Rad). Each sample was amplified using up to 30 cycles (20 s 94° C.; 20 s 60° C.; 20 s 72° C.) in a Bio-Rad iCycler iQ, and the data were analyzed using iCycler IQ version 3.0. The data were normalized using the reference genes PDA1 and ACT1 and the signal was scaled to mRNA copies/cell according to a SAGE study³⁹ in which copies of mRNA/cell of all the reference genes had previously been determined.

Polysome profiling 50 ml of 2×CBS media was inoculated with individual yeast colonies and cultured over the weekend in the presence or absence of 0.5 μg/ml doxycycline at 220 rpm and 30° C. These cultures were used to inoculate fresh 50 ml 2×CBS media to a final OD600 nm of 0.1 and were cultured as above for 24 hours. 50 ml 2×CBS was inoculated to a final OD600 nm of 0.1 from these cultures and grown as stated above until a final OD600 nm of 1 was obtained. Cyclohexemide was then added to the cultures to a final concentration of 10 μg/ml and incubated for 10 mins. The cells were then recovered at 5,000 g, 4° C., 5 mins and stored on ice.

Cells were resuspended in 0.4 ml of lysis buffer (10 mM Tris-HCl pH7.4, 100 mM NaCl, 30 mM MgCl₂, 200 ug/ml heparin, 50 μg/ml cycloheximide, 1 mM DTT, 1 μl/ml RNase inhibitor, EDTA-free protease inhibit cocktail 1 tablet in 10 ml buffer) prepared in fresh DEPC treated water and an equivalent volume of acid-washed glass beads added. The cells were then disrupted in a precellys 24 (Bertin Technology) at a speed of 6500 rpm for 10 secs. The samples were then cooled on ice for 2 mins and 2 additional rounds repeated. The samples were then clarified twice at 5,000 g, 4° C., 5 mins and 10,000 g, 4° C., 20 mins and the supernatant recovered. The RNA was subsequently quantified at 260 nm on a nanodrop (Thermo scientific) and/or treated with 50-100 mM EDTA and 5-10 OD units loaded on top of freshly prepared 10 ml 10-50% sucrose gradients for polysome profiling or 10 ml 7-30% sucrose gradients for ribosome disruption profiling. The gradients were then ultra-centrifuged at 37,000 rpm 4° C., in a SW 40 Beckman rotor for 2 hours 40 mins.

The OD₂ profile of the gradients were recorded by chart recorder (Pharmacia LKB REC 102) by passing the gradient (from the bottom of the tube to the top of the tube) through an AmershamPharmacia UV detector at a speed of 1.8 ml/min with the simultaneous collection of 0.7 ml fractions. These fractions were stored at −20° C. for later analysis.

Production of hA2aR and membrane preparation. BY4714 and yTHCBMS1 strains were transformed with pYX212 hA2aR and pYX222 hA2aR vectors respectively and cultured in 1.75 L of 2×CBS supplemented with 10 mM Theophylline and 0.5 μg/ml doxycycline as previously outlined. The cells were harvested by centrifugation once the glucose concentration of the cultures decreased to between 5-10 mM. The cells were re-suspended in 30 ml of breaking buffer and disrupted at 30,000 psi for 10 minutes using an EmulsiFlex-C3 (Avestin Inc.). The samples were clarified by centrifugation at 10,000 g, 4° C. for 30 min and total membranes recovered from the supernatant at 100,000 g, 4° C. for 60 min. Total membranes were re-suspended in 2.5 ml of Buffer A, the protein concentration determined using a Nanodrop and 0.5 ml aliquots stored at −80° C.

Radioligand binding assay. Membrane bound hA2aR was determined using a radioligand binding assay. Membranes at 0.5 mg/ml were incubated with varying concentrations of 3H ZM241385 for 60 minutes at 30 C and non-specific binding was defined by including 1 micromolar ZM241385 in the incubations. Assays were terminated by spinning at 14,000 rpm in a bench-top centrifuge for 5 minutes. The supernatant was discarded, the pellets washed superficially with water and solublilised with Soluene. This was added to scintillation fluid and then counted to determine bound radioactivity.

### Results

43 strains were initially screened in shake-flasks to test the effect of genes that we had previously shown to be down-regulated under high-yielding production conditions⁷ or that were known to be from related pathways, especially in cases where the deletion strain was non-viable. The amount of membrane-bound Fps1 from late-log phase shake-flask cultures was quantified from immunoblots relative to the wild-type yield (data not shown). Immunoblot signals were analyzed using the ImageGauge programme, and were below saturation (data not shown). A cut-off point 2-fold over wild-type was set for selecting strains for further analysis in fermentors

Three strains deleted for genes involved in transcriptional regulation give improved yields of Fps1. FIG. 1 summarises the Fps1 overproduction data in the three selected S. cerevisiae strains and demonstrates clearly that in bioreactors the spt3Δ strain offers a 9-69-fold medium-dependent increase in Fps1 yield compared with wild type, srb5Δ strain a 1-18-fold increase and gcn5Δ strain a 4-46-fold increase. The spt3Δ strain gave the highest final yield of 54-fold over our internal control. We noted from these data that the move from shake flask to bioreactor had resulted in significant yield improvements for each strain relative to the wild-type, with the greatest increase in yield compared to the wild-type strain being only 5-fold for the spt3Δ and srb5Δ strains in shake-flasks (data not shown). The choice of medium also had a role to play with the relationship between increased yield and medium composition being strain independent: 2×CBS was optimal in all cases (FIG. 1), although CSM media revealed greater improvements in the deletion strains compared to wild-type. The relationship between increased biomass and Fps1 yield was not correlated since the highest yield of Fps1 was observed in early-to-mid glucose phase samples, whereas the highest biomass was always achieved in the ethanol phase. All mutant strains grew to lower cell densities than the wild type and exhibited slow-growth phenotypes. The reported auxotrophy of srb5Δ for myo-inositol¹⁴, was not a significant contributor to its phenotype under our experimental conditions with it consistently out-performing the wild type in Fps1 overproduction (FIG. 1).

The three deletion strains do not give increased Fps1 yields on account of increased promoter activity—or FPS1 transcript number, but rather have up-regulation of BMS1 in common. To ascertain whether or not the increased yields of Fps1 in the three deletions strains was attributable to increased transcription of FPS1 we performed real time Q-PCR. The data was normalised using the reference genes ACT1 and PDA1 and the signal scaled to mRNA copies per cell according to the methodology of the SAGE study³⁹. In all cases no significant difference in the copy number of FPS1 was established in the deletion strains compared with wild-type (Table1). Subsequent real time Q-PCR demonstrated that the increased Fps1 yield of the deletion strains positively correlated with BMS1 transcript number. Table 1.

The spt3Δ strain, which gives the highest yields for Fps1 can also be used to improve the functional yield of the G-protein coupled receptor, hA2aR. In order to examine whether our screening strategy using Fps1 as a target, had resulted in an Fps1-specific result, we tested the G-protein coupled receptor, hA2aR in the best-performing strain. FIG. 3A shows that the improvement in active hA2aR was 4.2-fold, which was consistent with an increase in the immunoblots signal of 4.03-fold (data not shown). This suggested that the spt30 strain, at least, might be useful in increasing yields for a range of proteins with low to medium starting yields.

Maximum yields of Fps1 are achieved by tuning BMS1 transcript number in a doxycycline-repressible system. To investigate the positive relationship observed between the Fps1 yield and BMS1 we expressed Fps1 in a yTHCBMS1 strain. This strain has the endogenous BMS1 promoter replaced with a TET-titratable promoter in the genome. Thus, the expression of the gene can be attenuated by the addition of doxycycline to the yeast's growth medium. We have found that whilst there is no correlation between the amount of BMS1 transcript and Fps1 yield in a titration study, that the maximum yield of Fps1 is achieved by tuning the expression of BMS1 to approx 4-fold above wild-type level (data not shown). This in turn correlated to an 18-fold increase in the yield of Fps1 relative to the yield obtained with the spt3Δ strain (FIG. 2).

BMS1 overexpression improves the functional yield of hA2aR 3-fold and GFP 2-fold. Once again we verified whether the improved protein yield was an Fps1 only effect by expressing the G-protein coupled receptor, hA2aR and green fluorescent protein (GFP) in the yTHCBMS1 strain. We found that the improvement in active hA2aR was 3-fold compared with wild-type (data not shown) whilst the yield of GFP could be improved 2-fold (FIG. 3B). We noted from the data that the concentration of doxycycline yielding these improvements was different in each case from that previously observed for Fps1. Specifically for hA2aR we found that doxycycline at a concentration of 1 μg/ml in the yeast media resulted in a 3-fold improvement of the yield compared to wild-type, whilst a concentration of 10 μg/ml of doxycycline resulted in a 2-fold improvement of GFP compared with wild-type.

Maximum membrane protein yield is correlated with efficiency of translation as measured in polysome profiling experiments. Having established that the increased yields of Fps1 were not attributed to increased transcription we reasoned that a translational mechanism may account for the observed phenotypes. Thus we determined the polysome profile for both the yTHCBMS1 strain and wild-type under high (0.5 μg/ml doxycycline) and low (0 μg/ml doxycycline) Fps1 yielding conditions (FIG. 4A). We observed no difference in the wild-type profiles under the two conditions tested, whereas the yTHCBMS1 profiles exhibited significant differences. It can be seen that significantly more 60S and 40S is produced in the yTHCBMS1 strain compared with wild-type and that the amount of 40S decreases with an increasing doxycycline concentration (FIG. 4B) without a significant reduction in the amount of polysome and 80S fraction (FIG. 4A).

Maximum membrane protein yield is correlated with increased metabolic activity as measured by flow microcalorimetry. Calorimetric traces for the wild-type strain in the absence or presence of 0.5 μg/mL doxycycline and the BMS1 strain in its absence were all highly similar with pseudo-first order rate constants of 9×10⁻⁵ s¹, 1.09×10⁻⁴ s⁻¹ and 1.03×10⁻⁴ s⁻¹ respectively. In addition, it was clear that a second process, most likely the contribution to metabolic activity caused by protein production, did not have a significant contribution to the growth of the organism which had yielded the rate constants above. By subtracting the effect of the growth calculated from the three suboptimal expression conditions, it was possible to obtain a pseudo-first order rate constant, 6×10⁻⁵ s⁻¹ indicating that metabolic processes associated with protein production occur at approximately 60% of the rate of those associated with growth. FIG. 5 shows the thermodynamic profiles of both yTHCBMS1 and wild-type in the presence of 0.5 μg/ml doxycycline producing Fps1.

BMS1 overexpression can be used as a marker for selecting high-yielding strains. In order to investigate whether BMS1 overexpression can be used as a marker for identifying high-yielding recombinant protein production strains we analysed the number of copies of BMS1 mRNA per cell in 3 other strains identified from our original panel; spt8Δ, med6Δ, and yTHCSRB6. We found that only yTHCSRB6 had significantly increased levels of BMS1 and that this strain again correlated to an increased yield of Fps1 relative to wild-type (Table 2).

### BMS1 and SRB6 Expression

Analysis of Fps1 yields as assessed by immunoblotting in S. cerevisiae yeast cells are shown in comparison to wild type. Lowercase letters denote deletion strains while uppercase letters denote strains with upregulated expression of a particular strain. BMS1 and SRB6 are in bold as yields obtained for these strains exceeded twice that of wild type.

### Discussion

The inventors wanted to demonstrate that by understanding the critical parameters in a membrane protein production experiment it would be possible to use this information to develop new production strains. In our initial screen we identified three high-yielding deletion strains, spt3Δ, srb5Δ and gcn5Δ, where the deleted gene had an apparent role in transcriptional regulation. Within a yeast cell at any one promoter at any time, the transcriptional machinery might include the RNA polymerase II core enzyme, general transcription factors, the Swi/Snf complex, the SAGA complex and the mediator complex.

The SAGA (Spt-Ada-Gcn5 acetyltransferase) complex was recently modelled to approximately 30 Å¹³. This 1.8 MDa S. cerevisiae multi-subunit transcriptional co-factor, which comprises 14 polypeptides, was shown to be arranged into five distinct domains. It is a required for the acetylation of the octomeric histone scaffold of chromatin primarily through the Gcn5 catalytic subunit, located in domain III¹⁵⁻¹⁷ which acetylates H3 within the nucleosome, and is in turn mediated through the roles of Ada2 and Ada3^{18, 19}. SAGA can also regulate TATA-binding protein (TBP) interactions through the roles of Spt3, and Spt8, which are located in the flexible domain V¹³ at specific promoters including PHO5, GAL1, HIS3 and HO²⁰⁻²⁵. Spt3 has been shown by biochemical and genetic studies to interact with TBP^{23, 24} suggesting that it is structurally similar to TBP-associated factors (TAFs). Studies have indicated that Spt3 is generally required for the recruitment of TBP to specific promoters, whereas Spt8 is required at only a sub-set of SAGA-dependent promoters^{26, 27}. It has been suggested that SAGA may have a role in the transcription of 10% of genes, most of which seem to be stress-induced. This might reflect the need to balance inducible stress responses with the steady output of housekeeping genes²⁸.

In contrast, the mediator complex appears to be required for all transcriptional events. It is comprised of 21 core subunits in both its free form and as a holoenzyme with RNA polymerase II^{29, 30} It transmits regulatory signals from transcription factors to RNA polymerase II, interacting directly with the unphosphorylated carboxy terminal domain of RNA polymerase II, forming part of the pre-initiation complex, thus stimulating transcription. However the mediator can also associate with a subset of proteins (Srb8-11) which prevents its interaction with RNA polymerase II and negatively regulates a small set of genes. Mediator subunits can be assigned to three modules: head, middle and tail as identified by EM as well as to an additional kinase module^{31, 32}. Srb2 and Srb5 (also termed Med20 and Med18³³) are two of eight proteins that comprise the head module and whilst they are not essential for yeast viability, they are essential for the formation of a stable pre-initiation complex, efficient basal transcription and transcriptional activation. A comparison of the structure of Med18 elucidated from the head sub-complex Med8-Med18-Med20 structure³⁴ demonstrated a resemblance to phosphoryl transferase enzymes; however Med18 lacks a functional active site due to the absence of the conserved ExExK motif. Srb5 and Srb6 are part of the Srb4 sub-complex, which functions in the modulation of general polymerase activity.

The inventors have shown that individually deleting the 3 genes SPT3, SRB5 and GCN5 involved in the transcriptional complexes outlined above leads to a 6-7 fold upregulation of the BMS1 gene by an as yet uncharacterised route which would appear to account for the high-yielding phenotypes observed. In bioreactors they have demonstrated that spt3Δ offers a 9-69 fold increase in Fps1 yields, srb5Δ a 1-18 fold increase and gcn5Δ a 4-46 fold increase compared with wild-type. In addition they have managed to increase the functional yield of the human GPCR, adenosine 2a receptor 4.2 fold in the spt3Δ strain compared with wild-type.

Ribosomes are large macromolecular machines that catalyze protein synthesis in all cells. Groundbreaking work in bacteria has provided insight into the order of binding of ribosomal proteins to ribosomal RNA (rRNA) and has given a structural and thermodynamic rationale for this order. However, in eukaryotic cells the assembly process is much more complex, requiring a macromolecular machinery of >170 proteins and >70 RNAs. While the inventors know that this machinery is absolutely essential, they have little understanding of the function of the individual players. It is estimated that dividing yeast cells require 2000 ribosomes every minute with every ribosome being composed of 4 rRNAs and 79 ribosomal proteins³⁵. Both of the biosynthetic pathways governing ribosome biogenesis are therefore stringently regulated with the pathway associated with rRNA biosythesis reported to include over 200 genes^{35, 36} which includes BMS1. It is clear from the inventor's work that by stressing the host cell with recombinant protein production causes elements of the ribosome biosythesis pathway to contribute a fundamental role in subsequent recombinant protein translational efficiency.

Bms1 is an essential nucleolar protein that is evolutionarily conserved throughout the eukaryotic kingdom and has been suggested to have a regulatory role in the biogenesis of the 40S subunit³⁷ (the mRNA decoding subunit) as well as being a GTP-binding protein^{12, 13}. Further experiments have led to the current model of Bms1 binding to the product of a second essential gene, Rcl1, in a GTP-dependent manner and shuttling Rcl1 to pre-ribosomes via its affinity for U3 snoRNA¹³. Our unpublished data also suggest that over expression of Rcl1 in a tetO overexpression strain does not correlate with improved protein yields, which could be a result of Bms1 levels being limiting. The data suggests that translation is enhanced by overexpression of BMS1 with Fps1 yields up to 18-fold higher than those achieved in the spt3Δ, strain. Surprisingly the polysome profile for a strain in which BMS1 is overexpressed in the presence of optimal levels of doxycycline for maximal Fps1 yield (at 0.5 μg/mL, FIG. 4A) clearly shows elevated levels of 60S subunits without any significant decrease in the levels of 80S or polysome. Corresponding Northern blots (data not shown) show no difference in levels of FPS1 transcript between the strains, as previously seen in the Q-PCR experiments⁷. Furthermore when the ribosomal subunits are dissociated in the presence of EDTA (FIG. 4B), it is clear that in the BMS1 overexpression strain there is more of both the 60S and 40S subunits, with the relative amount of 40S decreasing with increasing doxycycline concentration (FIG. 4B). The inventors have also demonstrated that the functional yield of both GFP and hA2aR can be increased 2 and 3 fold respectively compared with wild-type in the yTHCBMS1 strain.

Recently, Wagner et al 2007³⁸ used a similar host response strategy to this to study membrane protein overexpression in E. coli which highlighted deficiencies in the host secretory pathway (specifically the Sec translocon). They found that the overexpression of three membrane proteins (YidC, YedZ, and LepI), but not soluble GST-GFP, resulted in accumulation of cytoplasmic aggregates containing the overexpressed proteins, chaperones (DnaK/J and GroEL/S), and soluble proteases (HsIUV and CIpXP) as well as many precursors of periplasmic and outer membrane proteins. This was consistent with lowered accumulation levels of secreted proteins in the three membrane protein overexpressors and is likely to be a direct consequence of saturation of the cytoplasmic membrane protein translocation machinery. Of note is that in our system, overexpression of Sec63 does not give improved yields of Fps1 which may indicate that the sec translocon is not such a critical parameter in yeast. This emphasises important differences in prokaryotic and eukaryotic translation; the former being relatively well-understood in stark contrast to the latter.

In summary 43 deletion strains were screened to test the effect of genes that the inventors had previously shown to be down-regulated under high-yielding production conditions⁷ or that were known to be from related pathways, especially in cases where the deletion strain was non-viable. A more detailed investigation of three deletion strains; spt3Δ, srb5Δ and gcn5Δ demonstrated improved yields of Fps1 up to 69-fold compared with wild-type. This high yielding phenotype was found not to be due to increased transcription of FPS1 but due to increased amounts of BMS1. Furthermore increased functional yields of the human GPCR adenosine 2a have been produced in the spt3Δ strain compared with wild-type. Subsequent investigation of a BMS1 overexpression strain revealed that by tuning the expression of this gene it is possible to optimise both recombinant soluble and membrane protein yields through an enhanced translational mechanism.

### Tables

TABLE 1
Fps1 yield correlates with BMS1 transcript number.

| | Copies mRNA/cell | | | |
|---|---|---|---|---|
| Strain | | TPI | FPS1 | BMS1 |

| | | | | | | |
|---|---|---|---|---|---|---|
| wild-type | 53.6 | (5.0) | 60.1 | (18.2) | 0.1 | (0.0) |
| gcn5Δ | 15.4 | (3.4)** | 61.5 | (3.0) | 0.7 | (0.2)** |
| spt3Δ | 12.5 | (1.8)** | 64.9 | (26.0) | 0.6 | (0.1)** |
| srb5Δ | 38.2 | (12.5)* | 87.4 | (1.5) | 0.7 | (0.2)** |

Analysis of mRNA was performed using real time Q-PCR, which yielded copies of mRNA/cell. The data were normalised using the reference genes ACT1 and PDA1 and the signal was scaled to mRNA copies/cell according to a SAGE study³⁹, in which copies of mRNA/cell of all the reference genes had previously determined. Standard deviation in parenthesis was calculated for the samples (n = 4). A single asterisk denotes significance at the P < 0.05 level (Paired 2-tailed T-test), and a double asterisk at P < 0.01 level.

TABLE 2
BMS1 overexpression can be used as a marker
for selecting high-yielding strains.

| | | |
|---|---|---|
| | Copies mRNA/ | Fps1 yield relative |
| Strain | cell of BMS1 | to wild-type |

| | | | | |
|---|---|---|---|---|
| wild-type | 0.1 | (0.1) | 100 | (27.0) |
| spt8Δ | 0.2 | (0.0) | 104 | (28.0) |
| med6Δ | 0.2 | (0.1) | 55.6 | (15.0) |
| yTHCSRB6 | 0.8 | (0.3)** | 310 | (12.0)** |

Analysis of mRNA was performed using real time Q-PCR, which yielded copies of mRNA/cell. The data were normalised using the reference genes ACT1 and PDA1 and the signal was scaled to mRNA copies/cell according to a SAGE study³⁹, in which copies of mRNA/cell of all the reference genes had previously been determined. Standard deviation in parentheses was calculated for the samples (n = 4). A double asterisk denotes significance at the P < 0.01 level (Paired 2-tailed T-test). SPT8 encodes a subunit of the SAGA complex, required for inhibition at some promoters. MED6 and SRB6 encode subunits of the mediator complex.

### REFERENCES

1. Karlsson, M. et al. Reconstitution of water channel function of an aquaporin overexpressed and purified from Pichia pastoris. FEBS Lett. 537, 68-72 (2003).
2. Long, S. B., Campbell, E. B. & Mackinnon, R. Crystal structure of a mammalian voltage-dependent Shaker family K+ channel. Science 309, 897-903 (2005).
3. Martinez Molina, D. et al. Structural basis for synthesis of inflammatory mediators by human leukotriene C4 synthase. Nature 448, 613-616 (2007).
4. Newstead, S., Kim, H., von Heijne, G., Iwata, S. & Drew, D. High-throughput fluorescent-based optimization of eukaryotic membrane protein overexpression and purification in Saccharomyces cerevisiae. Proc Natl Acad Sci USA 104, 13936-13941 (2007).
5. Jidenko, M. et al. Crystallization of a mammalian membrane protein overexpressed in Saccharomyces cerevisiae. Proc. Natl. Acad. Sci. USA 102, 11687-11691 (2005).
6. Toyoshima, C., Nakasako, M., Nomura, H. & Ogawa, H. Crystal structure of the calcium pump of sarcoplasmic reticulum at 2.6 A resolution. Nature 405, 647-655 (2000).
7. Bonander, N. et al. Design of improved membrane protein production experiments: quantitation of the host response. Protein Sci 14, 1729-1740 (2005).
8. Griffith, D. A., Delipala, C., Leadsham, J., Jarvis, S. M. & Oesterhelt, D. A novel yeast expression system for the overproduction of quality-controlled membrane proteins. FEBS Lett 553, 45-50 (2003).
9. Bill, R. M. Yeast-a panacea for the structure-function analysis of membrane proteins? Curr Genet. 40, 157-171 (2001).
10. Hedfalk, K. et al. A regulatory domain in the C-terminal extension of the yeast glycerol channel Fps1p. J Biol Chem 279, 14954-14960 (2004).
11. Tamas, M. J. et al. A short regulatory domain restricts glycerol transport through yeast Fps1p. J Biol Chem 278, 6337-6345 (2003).
12. Gelperin, D., Horton, L., Beckman, J., Hensold, J. & Lemmon, S. K. Bms1p, a novel GTP-binding protein, and the related Tsrlp are required for distinct steps of 40S ribosome biogenesis in yeast. Rna-a Publication of the Rna Society 7, 1268-1283 (2001).
13. Karbstein, K. & Doudna, J. A. GTP-dependent formation of a ribonucleoprotein subcomplex required for ribosome biogenesis. Journal of Molecular Biology 356, 432-443 (2006).
14. Thompson, C. M., Koleske, A. J., Chao, D. M. & Young, R. A. A Multisubunit Complex-Associated with the Rna Polymerase-Ii Ctd and Tata-Binding Protein in Yeast. Cell 73, 1361-1375 (1993).
15. Shim, H. et al. c-Myc transactivation of LDH-A: implications for tumour metabolism and growth. Proc Natl Acad Sci USA 94, 6658-6663 (1997).
16. Brownell, J. E. et al. Tetrahymena histone acetyltransferase A: A homolog to yeast Gcn5p linking histone acetylation to gene activation. Cell 84, 843-851 (1996).
17. RuizGarcia, A. B., Sendra, R., Pamblanco, M. & Tordera, V. Gcn5p is involved in the acetylation of histone H3 in nucleosomes. Febs Letters 403, 186-190 (1997).
18. Balasubramanian, R., Pray-Grant, M. G., Selleck, W., Grant, P. A. & Tan, S. Role of the Ada2 and Ada3 transcriptional coactivators in histone acetylation. Journal of Biological Chemistry 277, 7989-7995 (2002).
19. Sterner, D. E., Wang, X., Bloom, M. H., Simon, G. M. & Berger, S. L. The SANT domain of Ada2 is required for normal acetylation of histones by the yeast SAGA complex. Journal of Biological Chemistry 277, 8178-8186 (2002).
20. Barbaric, S., Reinke, H. & Horz, W. Multiple mechanistically distinct functions of SAGA at the PHO5 promoter. Molecular and Cellular Biology 23, 3468-3476 (2003).
21. Belotserkovskaya, R. et al. Inhibition of TATA-binding protein function by SAGA subunits Spt3 and Spt8 at Gcn4-activated promoters. Molecular and Cellular Biology 20, 634-647 (2000).
22. Bhaumik, S. R. & Green, M. R. SAGA is an essential in vivo target of the yeast acidic activator Gal4p. Genes & Development 15, 1935-1945 (2001).
23. Eisenmann, D. M., Arndt, K. M., Ricupero, S. L., Rooney, J. W. & Winston, F. Spt3 Interacts with Tfiid to Allow Normal Transcription in Saccharomyces-Cerevisiae. Genes & Development 6, 1319-1331 (1992).
24. Larschan, E. & Winston; F. The S. cerevisiae SAGA complex functions in vivo as a coactivator for transcriptional activation by Ga14. Genes & Development 15, 1946-1956 (2001).
25. Brown, B. J., Hyun, J. W., Duvvuri, S., Karplus, P. A. & Massey, V. The role of glutamine 114 in old yellow enzyme. J Biol Chem 277, 2138-2145 (2002).
26. Bhaumik, S. R. & Green, M. R. Differential requirement of SAGA components for recruitment of TATA-box-binding protein to promoters in vivo. Molecular and Cellular Biology 22, 7365-7371 (2002).
27. Dudley, A. M., Rougeulle, C. & Winston, F. The Spt components of SAGA facilitate TBP binding to a promoter at a post-activator-binding step in vivo. Genes & Development 13, 2940-2945 (1999).
28 Huisinga, K. L. Pugh, B. F. A Genome-Wide Housekeeping Role for TFIID and a Highly. Regulated Stress-Related Role for SAGA in Saccharomyces cerevisiae. Mol Cell 13 (4) 573-583 (2004)
29. Kim, Y. J., Bjorklund, S., Li, Y., Sayre, M. H. & Kornberg, R. D. A Multiprotein Mediator of Transcriptional Activation and Its Interaction with the C-Terminal Repeat Domain of Rna-Polymerase-Ii. Cell 77, 599-608 (1994).
30. Myers, L. C. et al. The Med proteins of yeast and their function through the RNA polymerase II carboxy-terminal domain. Genes & Development 12, 45-54 (1998).
31. Dotson, M. R. et al. Structural organization of yeast and mammalian mediator complexes. Proceedings of the National Academy of Sciences of the United States of America 97, 14307-14310 (2000).
32. Kang, J. S. et al. The structural and functional organization of the yeast mediator complex. Journal of Biological Chemistry 276, 42003-42010 (2001).
33. Bourbon, H. M. et al. A unified nomenclature for protein subunits of Mediator complexes linking transcriptional regulators to RNA polymerase II. Molecular Cell 14, 553-557 (2004).
34. Lariviere, L. et al. Structure and TBP binding of the Mediator head subcomplex Med8-Med18-Med20. Nature Structural & Molecular Biology 13, 895-901 (2006).
35 Wade, C. H., Shea, K. A., Jensen, R. V., McLlear, M. A. EBP2 is a member of the yeast RRB regulon, a transcriptionally coregulated set of genes that are required for ribosome and rRNA biosynthesis. Mol. Cell. Biol. 21 (24) 8638-8650 (2001)
36 Wade, C. H., Umbarger, M. A., McAlear, M. A. The budding yeast rRNA and ribosome biosynthesis (RRB) regulon contains over 200 genes Yeast 23 (4) 293-306 (2006)
37 Wegierski, T., Billy, E., Nasr, F. & Filipowicz, W. Bms1p, a G-domain-containing protein, associates with Rcl1p and is required for 18S rRNA biogenesis in yeast. Rna-a Publication of the Rna Society 7, 1254-1267 (2001).
38 Wagner, S. et al Consequences of membrane protein overexpression in Escherichia coli. Mol Cell Proteomics 6, 1527-1550 (2007).
39. Velculescu, V. E. et al. Characterization of the yeast transcriptome. Cell 88, 243-251 (1997).

1. A eukaryotic cell comprising an upregulated BMS1 gene or a functional fragment thereof, and a nucleotide sequence encoding a recombinant protein or fragment thereof, for the production of the recombinant protein or fragment thereof.

2. A eukaryotic cell according to claim 1, wherein the upregulated BMS1 gene is upregulated by one or more of:
(i) a mutation in regulatory sequence for the BMS1 gene
(ii) providing one or more additional BMS1 genes in the cell compared to a wild type eukaryotic cell of the same species;
(iii) providing in the cell one or more BMS1 genes, operatively linked to a constitutive promoter; and/or
(iv) providing in the cell one or more BMS1 genes operatively lined to an inducible promoter.

3. A eukaryotic cell according to claim 1, wherein the cell is selected to have a level of BMS1 protein optimised for production of the protein or a fragment thereof by the cell.

4. A eukaryotic cell according to claim 1, additionally comprising an upregulated SRB6 gene or a functional fragment thereof.

5. A eukaryotic cell according to claim 1 selected from a yeast cell, a plant cell, an insect cell, an avian cell, or a mammalian cell.

6. A eukaryotic cell according to claim 5, selected from Saccharomyces cerevisiae, Schizosaccharomyces pombi and Pichia pastoris.

7. A eukaryotic cell according to claim 1 comprising a BMS1 gene operatively linked to a tet0 promoter.

8. A eukaryotic cell according to claim 1, wherein the protein is a transmembrane protein or a fragment thereof.

9. A eukaryotic cell according to claim 8, wherein the protein is a G-protein coupled receptor (GPCR).

10. A method of producing a recombinant protein or a fragment thereof from a nucleic acid sequence encoding the recombinant protein or fragment, comprising providing a eukaryotic cell having upregulated BMS1 and expressing the nucleic acid sequence encoding the recombinant protein, or a fragment thereof, in the cell.

11. A method according to claim 10 comprising the step of optimising the concentration of BMS1 in the cell.

12. A method according to claim 10, wherein the eukaryotic cell is a eukaryotic cell according to claim 1.

13. A method of testing a eukaryotic cell as a candidate cell for the production of a recombinant protein, comprising the step of measuring BMS1 expression in the cell.

14. A method according to claim 10, wherein the cell also comprises an upregulated SRB6 gene.

15. A eukaryotic cell for use in a method according to claim 10 comprising a BMS1 gene, or a functional fragment thereof, operatively attached to a heterologous constituitive or heterologous inducible promoter.

16. A kit for the production of recombinant protein or fragment thereof comprising (i) a vector encoding BMS1 or a functional fragment thereof and (ii) a nucleotide sequence encoding the recombinant protein or a fragment thereof.

17. A eukaryotic cell according to claim 15, comprising an SRB6 gene or functional fragment thereof, operatively attached to a heterologous constituitive or heterologous inducible promoter.

18. A kit for the production of a recombinant protein or a fragment thereof comprising a eukaryotic cell as defined in claim 1.

19. A kit according to claim 16, comprising an SRB6 gene or functional fragment thereof, operatively attached to a heterologous constituitive or heterologous inducible promoter.
